(19) United States
(12) Patent Application Publication
(10) Pub. No.: US 2023/0132433 A1
(43) Pub. Date: May 4, 2023

(54) LYOPHILISATION PROCESS

(71) Applicant: 4D Pharma León S.L.U., Leon (ES)

(72) Inventors: Sophie Declomesnil, Leeds (GB); Christophe Carite, Leeds (GB); Laura De Castro Franco, Leeds (GB)

(21) Appl. No.: 17/842,843

(22) Filed: Jun. 17, 2022

Related U.S. Application Data
(63) Continuation of application No. PCT/EP2020/087319, filed on Dec. 18, 2020.

(30) Foreign Application Priority Data
Dec. 20, 2019 (EP) 19383175.7
Aug. 6, 2020 (EP) 20382728.2

(51) Int. Cl. A61K 9/19; A61K 35/74

(52) U.S. Cl. CPC A61K 9/19; A61K 35/74

(57) ABSTRACT

The invention relates to processes and apparatus for lyophilising bacterial products which minimise the loss of viable cells during this process, enabling those products to be lyophilised in an efficient and economic manner. The lyophilisation process of the invention achieves improved viability of the lyophilised cells.

### CROSS-REFERENCE

This application is a continuation of International Application No. PCT/EP2020/087319, filed Dec. 18, 2020, which claims the benefit of European Application No. 19383175.7, filed Dec. 20, 2019, and European Application No. 20382728.2, filed Aug. 6, 2020, all of which are hereby incorporated by reference in their entirety.

### FIELD OF THE INVENTION

The present invention relates to processes and apparatus for lyophilising bacterial products which minimise the loss of viable cells during this process, enabling those products to be lyophilised in an efficient and economic manner.

### BACKGROUND TO THE INVENTION

Lyophilisation is a widely used process for formulating pharmaceutical, biotechnological and other types of product. It is an effective way to prepare solid products, even if those products are pharmaceutical products which are destined to be administered in liquid form to patients. Lyophilisation is also a convenient way to produce preparations containing live organisms, or chemically sensitive products obtained from organisms.

While there are many commercially operated lyophilisation processes, these typically involve three phases, namely i) freezing, ii) primary drying or sublimation and iii) secondary drying or desorption.

During the freezing phase and optionally also during the sublimation phase, the product is frozen, typically between −20° C. and −80° C. Once the product has attained the target reduced temperature, the pressure to which it is exposed is reduced, and a moderate amount of heat is applied, which causes the frozen water present in the product to sublime. This first step of the lyophilisation process generally results in the majority of the water present in the product being removed.

In the third phase, desorption, the temperature is increased to remove any non-frozen water molecules present in the product.

Generally speaking, effectively operated lyophilisation processes can be used to produce products having a very low water content, e.g. less than 5%.

On a pilot scale or on an industrial scale, lyophilisation is commonly carried out in freeze dryers. Freeze dryers conventionally include the following components: a) a vacuum pump to reduce the pressure in the dryer; and b) a condenser to remove moisture from the freeze dryer by condensation. Differences exist between freeze dryers in terms of how the product to be lyophilised is arranged.

In freeze dryers conventionally used in the preparation of pharmaceutical or biotechnological products, the product to be lyophilised may be loaded into the freeze dryer in bulk. In such arrangements, the bulk product is placed in trays and the trays are then loaded into the freeze dryer for lyophilisation.

The trays are generally shaped to maximise the surface area of the product which is exposed to the interior of the freeze dryer to facilitate sublimation and desorption of water from the product.

While trays of this type have been used effectively for many years, their use is not appropriate for the preparation of all types of product.

One problem that has been identified with the lyophilisation of live organisms is that the lyophilisation process is harsh and can result in unacceptable losses of viable cells. While, for some types of products, operators have sought to minimise such losses through optimisation of the lyophilization cycle and/or of the lyoprotectant formulation (if employed), the success of such approaches differs depending on the type of live cells which are undergoing freeze drying.

A class of live cells that have proven to be particularly challenging to lyophilise without an unacceptable loss of viable cells are anaerobic bacteria, particularly obligate anaerobic bacteria. One potential cause of this loss of viability was thought to be residual oxygen present in the lyophilisation apparatus and which came into contact with the lyophilisation medium during lyophilisation.

While purging conventional lyophilisation apparatus to render its interior anaerobic has been considered, the practicalities of doing so are challenging, especially for commercial scale apparatus, owing to the large interior volume and complexity of that apparatus. Additionally, purging of the apparatus via the application of vacuum at the initiation of a freeze drying cycle has been found to damage the product being subjected to lyophilisation in some instances.

Therefore, there remains a need in the art for processes for freeze drying anaerobic bacteria, especially anaerobic bacteria, on a large scale which do not result in an unacceptable loss in viable cells.

### SUMMARY OF THE INVENTION

Thus, according to a first aspect of the present invention, there is provided a process for preparing a lyophilised product comprising the steps of:

providing a lyophilisation medium comprising anaerobic bacteria under anaerobic conditions,

freezing the lyophilisation medium under anaerobic conditions to obtain a frozen lyophilisation medium,

conducting a sublimation step on the frozen lyophilisation medium

collecting a lyophilised product.

As explained above, freezing and sublimation steps are routine in freeze drying processes. However, the inventors have now unexpectedly found that by conducting the initial freezing step under anaerobic conditions, this renders the anaerobic bacterial cells less susceptible to oxygen induced inactivation. This means that, advantageously, the loss of viable anaerobic cells is reduced as compared to corresponding processes in which the freezing step is not conducted under anaerobic conditions. A further benefit of this process is that it reduces the operator burden on minimising oxygen levels in the lyophilisation apparatus; modest levels of oxygen within the freeze dryer can be tolerated.

The inventors have found and the examples demonstrate that freezing the lyophilisation medium under anaerobic conditions prior to or as part of conventional lyophilisation processes provides the significant benefit of effectively maintaining the viability of the bacterial cells present in the lyophilisation medium. In embodiments of the invention, the lyophilisation medium provided as part of the process of the present invention is maintained under anaerobic conditions until the step of freezing the lyophilisation medium is complete.

### DETAILED DESCRIPTION OF THE INVENTION

### Anaerobic Bacteria for Lyophilisation

The examples which follow also demonstrate that the process of the invention can be used to prepare lyophilised formulations comprising obligate anaerobic bacteria. Those skilled in the art will recognise that obligate anaerobes are bacteria that do not possess the defences that make aerobic life possible and therefore cannot survive in environments with even low to moderate levels of oxygen. Bacterial tolerance to oxygen is related to the ability of the bacterium to detoxify superoxide and hydrogen peroxide, produced as by-product of aerobic respiration. The assimilation of glucose in aerobic environments results in the terminal generation of free radical superoxide (O²⁻). The superoxide is reduced by the enzyme superoxide dismutase to oxygen gas and hydrogen peroxide (H₂O₂). Subsequently, the toxic hydrogen peroxide generated in this reaction is converted to water and oxygen by the enzyme catalase, which is found in aerobic and facultative anaerobic bacteria, or by various peroxidases which are found in several aerotolerant anaerobes.

The skilled person will recognise that among anaerobes, there are a number of sub-populations of bacteria characterised by their ability to process, and thus survive in the presence of atmospheric oxygen. As mentioned above, facultative and aerotolerant anaerobes possess the molecular machinery to process oxygen (albeit, in some strains, only in relatively modest levels) to non-toxic by-products. However, for obligate anaerobes, this machinery is not present or is incapable of processing anything more than very low levels of oxygen. Any type of anaerobic bacteria may be lyophilised according to the process of the invention. In preferred embodiments, the bacteria to be lyophilised in the process of the invention are obligate anaerobes.

Examples of aerotolerant anaerobic bacteria which may be lyophilised according to the process of the present invention include those belonging to the Streptococcus, Clostridium and Lactobacillus genera.

Examples of facultative anaerobic bacteria which may be lyophilised according to the process of the invention include those belonging to the genera:
Enterococcus (e.g Enterococcus gallinarum, Enterococcus caselliflavus, Enterococcus faecalis, or Enterococcus faecium), and
Pediococcus (e.g. Pediococcus acidilacticii)

Examples of obligate anaerobic bacteria which may be lyophilised according to the process of the invention include those belonging to the genera:
Roseburia (e.g. Roseburia hominis, Roseburia intestinalis or Roseburia inulinivorans),
Bacteroides (e.g. Bacteroides thetaiotaomicron, Bacteroides massiliensis, Bacteroides fragilis, Bacteroides ovatus, Bacteroides vulgatus, Bacteroides dorei, Bacteroides uniformis or Bacteroides copricola),
Bifidobacterium (e.g. Bifidobacterium breve, Bifidobacterium adolescentis or Bifidobacterium longum),
Parabacteroides (e.g. Parabacteroides distasonis, Parabacteroides goldsteinii,
Parabacteroides merdae, or Parabacteroides johnsonii), Eubacterium (e.g. Eubacterium contortum, Eubacterium fissicatena, Eubacterium limosum, Eubacterium eligens, Eubacterium hadrum, Eubacterium hallii, or Eubacterium rectale),
Faecalibacterium (e.g. Faecalibacterium prausnitzii),
Bariatricus (e.g. Bariatricus massiliensis),
Megasphaera (e.g. Megasphaera massiliensis),
Flavonifractor (e.g. Flavonifractor plautii),
Anaerotruncus (e.g. Anaerotruncus colihominis),
Ruminococcus (e.g. Ruminococcus torques, Ruminococcus gnavus, or Ruminococcus bromii),
Pseudoflavonifractor (e.g. Pseudoflavonifractor capillosus),
Clostridium (e.g. Clostridium nexile, Clostridium hylemonae, Clostridium butyricum, Clostridium tedium, Clostridium disporicum, Clostridium bifermentans, Clostridium inocuum, Clostridium mayombei, Clostridium bolteae, Clostridium bartletti, Clostridium symbiosum or Clostridium orbiscindens),
Coprococcus (e.g. Coprococcus comes, or Coprococcus cattus),
Acetivibrio (e.g. Acetovibrio ethanolgignens),
Dorea (e.g. Dorea longicatena)
Blautia (e.g. Blautia hydrogenotrophica, Blautia stercoris, Blautia wexlerae or Blautia producta), and
Erysipelatoclostridium (e.g. Erysipelatoclostridium ramosum).

In certain embodiments, the lyophilisation medium may comprise more than one bacterial strain (such as a consortium of different bacterial strains). In such embodiments, the lyophilisation medium may comprise at least 2, at least 3, at least 4, at least 5, at least 6, at least 7, at least 8, at least 9, at least 10, at least 11, at least 12, at least 13, at least 14 or at least 15 different bacterial strains. Additionally or alternatively, the lyophilisation medium may comprise 50 or less, 40 or less, 30 or less or 20 or less different bacterial strains. In certain embodiments, the consortium contains only obligate anaerobic bacteria. In certain embodiments, the consortium contains only facultative or aerotolerant anaerobic bacteria. In certain embodiments, the consortium contains both obligate and facultative or aerotolerant anaerobic bacteria.

In a preferred embodiment, the process of the invention may be used to lyophilise obligate anaerobic bacteria belonging to the genera: Akkermansia, Roseburia, Bacteroides, Parabacteroides, Blautia, Megasphaera and/or Blautia. In a preferred embodiment, the process of the invention may be used to lyophilise obligate anaerobic bacteria belonging to the species: Akkermansia muciniphila, Roseburia hominis, Bacteroides sp, Bacteroides thetaiotaomicron, Parabacteroides distasonis, Blautia stercoris, Megasphaera massiliensis and/or Blautia hydrogenotrophica. In a preferred embodiment, the process of the invention may be used to lyophilise obligate anaerobic bacteria belonging to the species: Akkermansia muciniphila, Roseburia hominis, Bacteroides thetaiotaomicron, Parabacteroides distasonis, Blautia stercoris, Megasphaera massiliensis and/or Blautia hydrogenotrophica. As shown in the examples, a process according to the present invention is suitable for providing viable lyophilised products of obligate anaerobic bacterial strains from Akkermansia muciniphila, Roseburia hominis, Bacteroides sp, Bacteroides thetaiotaomicron, Parabacteroides distasonis, Blautia stercoris, Megasphaera massiliensis and Blautia hydrogenotrophica.

Those skilled in the art will be familiar with apparatus and techniques for providing anaerobic conditions. For the avoidance of any doubt, as used herein, the term ‘anaerobic conditions’ is used to mean an environment in which oxygen levels are maintained below about 1000 ppm, below about 500 ppm, below about 200 ppm, below about 100 ppm, below about 50 ppm, below about 20 ppm, below about 10 ppm, below about 5 ppm, below about 2 ppm or below about 1 ppm. An assessment of oxygen content of an environment may be performed using a digital oxygen analyser, marketed under model number #800-DOI by Plas-Labs, Inc., Lansing, Mo., USA.

Those skilled in the art will also be familiar with the typical compositions of lyophilisation media and how they may be prepared. In embodiments of the invention, the lyophilisation medium comprises the anaerobic bacteria in the form of a concentrated biomass. In such embodiments, the biomass may be stored under anaerobic conditions from the time it is harvested (e.g. from a fermenter) until the completion of the freezing step employed in the process of the present invention.

In embodiments of the invention, the lyophilisation medium may comprise a lyobuffer. The lyobuffer may comprise excipients known to those of skill in the art, for example:

cryoprotectants (e.g. polyol such as ethylene glycol, sorbitol, propylene glycol, and/or glycerol; DMSO; skim milk; yeast extract; bovine serum albumin (BSA); starch hydrolysates; saccharides (including monosaccharides, disaccharides and/or polysaccharides) such as glucose, maltose, maltotriose, trehalose, mannitol, dextran, maltodextrin, lactose and/or sucrose; and/or amino acids such as cysteine, glutamic acid (optionally in the form of a salt, such as sodium glutamate), arginine and/or glycine),

antioxidants (e.g cysteine, arginine, ascorbic acid (and salts and esters thereof e.g. ascorbyl palmitate, sodium ascorbate)), butylated agents such as butylated hydroxyanisole or butylated hydroxytoluene, citric acid, erythorbic acid, fumaric acid, glutamic acid, glutathione, malic acid, methionine, monothioglycerol, pentetic acid, metabisulfite (such as sodium metabisulfite, potassium metabisulfite), propionic acid, propyl gallate, uric acid, sodium formaldehyde sulfoxylate, sulphite (e.g. sodium sulphite), sodium thiosulfate, sulphur dioxide, thymol, tocopherol (free or esterified), uric acid (and salts thereof) and salts and/or esters thereof),

bulking agents (e.g. mannitol, maltodextrin and/or glycine),

buffers (e.g. phosphate, citrate, tris and/or Hepes), and/or

surfactants (e.g. polysorbate (such as those commercialised under the trade mark Tween)) and/or sorbitan (such as those commercialised under the trade mark Span).

In some embodiments, the lyobuffer does not comprise inulin. In some embodiments, the lyobuffer does not comprise cysteine. In some embodiments, the lyobuffer does not comprise inulin and cysteine. In some embodiments, the lyobuffer does not comprise inulin and riboflavin. In some embodiments, the lyobuffer does not comprise inulin, cysteine and riboflavin.

In a particular embodiment, the lyobuffer comprises an excipient formula comprising (at a final concentration prior to lyophilisation): 2% sucrose, 4% maltodextrine DE9 and 0.2% cysteine HCl. In a preferred embodiment, the ratio of biomass to excipients is about 70:30. In some embodiments, the lyobuffer does not comprise trehalose. In some embodiments, the lyobuffer does not comprise maltodextrine. In some embodiments, the lyobuffer does not comprise maltodextrine DE9. In some embodiments, the lyobuffer comprises (at a final concentration prior to lyophilisation): 2% sucrose and 0.2% cysteine HCl.

In some embodiments, the lyobuffer comprises excipients to protect the anaerobic bacteria during lyophilisation or to provide functional properties to the lyophilizate. Examples of excipients that can be present in the lyophilizate include mannitol, skim milk and bovine serum albumin (BSA), sucrose, trehalose and/or one of the other sugars identified above. A mixture of mannitol and sucrose as lyobuffer may be used.

In some embodiments, the lyobuffer comprises an antioxidant, (e.g. cysteine or a salt thereof).

In some embodiments, the antioxidant can be present as a salt. Examples of salts can include acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

### The Filling Step

The step of freezing the lyophilisation medium can be carried out in any type of apparatus provided that anaerobic conditions are maintained. Advantageously, the process of the invention is applicable to conventional lyophilisation apparatus.

In embodiments, the process of the invention comprises the step of filling the lyophilisation medium into a receptacle under anaerobic conditions. This step may be performed before or after the step of freezing the lyophilisation medium. Additionally or alternatively, the step of filling the lyophilisation medium into a receptacle under anaerobic conditions may be carried out in the same apparatus, or a different apparatus, as the step of freezing the lyophilisation medium, e.g. the filling step could be carried out in filling apparatus and the freezing step could be carried out in a lyophilisation apparatus or in a separate freezing apparatus.

The receptacle into which the lyophilisation medium is filled may be a tray, for example a conventional open tray or a specialist tray such as those commercialised by Gore® under the trade mark Lyogard®. Alternatively, a lyophilisation bag, such as that disclosed in International Patent Application No. PCT/IB2018/055246 (the contents of which are incorporated by reference herein) may be employed in the filling step.

In embodiments of the invention, the receptacle may be oxygen impermeable to facilitate the maintenance of anaerobic conditions therein. As used herein, the term ‘oxygen impermeable’ is used to identify a receptacle as having an oxygen transmission rate (OTR) of about 10 cc/m²/24 hrs or less, about 5 cc/m²/24 hrs or less, about 1 cc/m²/24 hrs or less, about 0.5 cc/m²/24 hrs or less, about 0.1 cc/m²/24 hrs or less, about 0.05 cc/m²/24 hrs or less, about 0.01 cc/m²/24 hrs or less, about 0.005 cc/m²/24 hrs or less or about 0.001 cc/m²/24 hrs or less as measured using a coulometric sensor operated in accordance with ASTM D3985. In embodiments of the invention, the receptacle has an oxygen transmission rate (OTR) of about 1 cc/m²/24 hrs or less. In certain embodiments of the invention, the receptacle has an oxygen transmission rate (OTR) of about 0.1 cc/m²/24 hrs or less. In preferred embodiments of the invention, the receptacle has an oxygen transmission rate (OTR) of about 0.01 cc/m²/24 hrs or less.

In such embodiments the process of the invention may comprise the step of closing the receptacle (e.g. lyophilisation bag) following the filling step to form a oxygen impermeable seal, thus providing a oxygen impermeable sealed receptacle. The seal may be provided by providing a closure to an opening (e.g. a filling port) comprised in the receptacle and/or by sealing (e.g. heat sealing) portions of the wall of the receptacle. In certain embodiments, the oxygen impermeable sealed receptacle is a lyophilisation bag.

Preferably, in such embodiments, the filling and closing steps are carried out before the step of freezing the lyophilisation medium. Such an approach is advantageous as it permits the freezing of the lyophilisation medium to occur under anaerobic conditions even where the apparatus in which the freezing step is carried out is not operated under anaerobic conditions. In other words, the freezing step could be performed under anaerobic conditions in conventional lyophilisation apparatus, the interior of which does not require special treatment to render it anaerobic. I

In embodiments in which the lyophilisation medium is filled into oxygen impermeable receptacles prior to the freezing step being carried out, the filled receptacles may be stored prior to the freezing step. Storage prior to the freezing step may be under anaerobic conditions and/or be under a temperature of 0° C. to about 10° C., or about 2° C. to about 8° C. In embodiments in which the filled, oxygen impermeable receptacles are closed, then the filled, oxygen impermeable receptacles may be stored under anaerobic conditions or non-anaerobic conditions.

Alternatively, the receptacle may be oxygen permeable. In such embodiments, the filling step may be carried out after the step of freezing the lyophilisation medium. This order of steps advantageously permits the use of conventional oxygen permeable receptacles in conventional lyophilisation apparatus without an unacceptable loss in bacterial viability owing to the finding that bacterial inactivation by oxygen exposure is significantly lower when said bacteria are frozen.

In alternative embodiments, the lyophilisation medium may be filled into oxygen permeable receptacles and the lyophilisation medium frozen, all under anaerobic conditions.

Anaerobic conditions during the filling step may be maintained using any apparatus or techniques known to those skilled in the art. For example, the filling step may be carried out in an anaerobic isolator, chamber or hood. In such embodiments, the anaerobic isolator, chamber or hood may be provided with a disposable liner such as that disclosed in International Patent Application No. PCT/IB2018/054749 (the contents of which are incorporated by reference).

In embodiments of the invention, the receptacle may be purged of oxygen prior to or during being filled.

### The Freezing Step

In the lyophilisation process of the invention, the initial freezing step is carried out under anaerobic conditions. The step of freezing the lyophilisation medium under anaerobic conditions can be conducted using any techniques or apparatus known to those skilled in the art. For example, the freezing step may be carried out in lyophilisation apparatus capable of being operated under anaerobic conditions which may optionally comprise freezing apparatus capable of being operated under anaerobic conditions.

In alternative arrangements, the step of freezing the lyophilisation medium under anaerobic conditions may be carried out using freezing apparatus not comprised within a lyophilisation apparatus. Such apparatus may be a cooling reactor (e.g. a cryogenic reactor) or a snap freezer.

In embodiments of the invention, following provision of the lyophilisation medium, the lyophilisation medium may be maintained under anaerobic conditions until completion of the step of freezing the lyophilisation medium.

In certain embodiments of the invention, during the step of freezing the lyophilisation medium, the lyophilisation medium may be exposed to a temperature of about −50° C. or lower, about −70° C. or lower, or about −90° C. or lower. In certain embodiments of the invention, during the step of freezing the lyophilisation medium, the lyophilisation medium may be exposed to a temperature of about −130° C. or higher, about −150° C. or higher, or about −200° C. or higher. For example, in certain embodiments, during the freezing step, the lyophilisation medium may be exposed to a temperature of about −50° C. to about −200° C. For example, in certain embodiments, during the freezing step, the lyophilisation medium may be exposed to a temperature of about −70° C. to about −150° C. For example, in certain embodiments, during the freezing step, the lyophilisation medium may be exposed to a temperature of about −90° C. to about −130° C. In certain embodiments, the freezing step may last for about 5 minutes or more, about 10 minutes or more, about 20 minutes or more, about 30 minutes or more about 60 minutes or more. In certain embodiments, the freezing step may last for about 600 minutes or less, about 300 minutes or less about 240 minutes or less or about 180 minutes or less. For example, in certain embodiments, the freezing step may last for about 5 minutes to about 600 minutes. For example, in certain embodiments, the freezing step may last for about 10 minutes to about 300 minutes. For example, in certain embodiments, the freezing step may last for about 20 minutes to about 240 minutes. For example, in certain embodiments, the freezing step may last for about 30 minutes to about 180 minutes. Such a freezing step may be conducted in lyophilisation apparatus or in separate freezing apparatus.

In embodiments of the invention, the step of freezing the lyophilisation medium is a snap freezing step. In certain embodiments, the snap freezing step involves the snap cooling of the lyophilisation medium in a freezing apparatus, e.g. liquid nitrogen-cooled freezer (for example to −110° C. for two hours).

In certain embodiments, the temperature to which the lyophilisation medium is exposed may be varied during the freezing step. For example, the lyophilisation medium may be exposed to a first temperature (e.g. about 20° C., about 10° C. or about 0° C. to about −20° C., about −30° C., about −40° C. or about −50° C.) and maintained at that temperature for a first period (for example about 1 minute, about 5 minutes or about 10 minutes to about 30 minutes, about 60 minutes, about 90 minutes or about 120 minutes) and then cooled to a second, lower temperature (for example those proposed in the preceding paragraph) and maintained at that second temperature for a second period (e.g. about 10 minutes, about 20 minutes, about 30 minutes or about 60 minutes to about 120 minutes, 180 minutes, 240 minutes, 300 minutes or longer). In such embodiments, the second period may be longer than the first period.

The step of freezing the lyophilisation medium may be carried out at atmospheric pressure. Alternatively, sub-atmospheric or supra-atmospheric pressure may be employed. In embodiments of the invention, atmospheric, or mildly sub- or supra-atmospheric pressures are preferred. For example, in certain embodiments, the pressure employed during the freezing step is about 10 kPa below atmospheric pressure to about 10 kPa above atmospheric pressure. In certain embodiments, the pressure employed during the freezing step is about 5 kPa below atmospheric pressure to about 5 kPa above atmospheric pressure. In certain embodiments, the pressure employed during the freezing step is about 2 kPa below atmospheric pressure to about 2 kPa above atmospheric pressure.

### The Sublimation Step

The step of freezing the lyophilisation medium under anaerobic conditions may be carried out in the same or different apparatus as that in which the sublimation step is carried out.

In some embodiments, the step of freezing the lyophilisation medium under anaerobic conditions and the sublimation step may be carried out in the same apparatus. For example, in arrangements in which the lyophilisation medium has been filled under anaerobic conditions into an oxygen impermeable receptacle which is then closed to form an oxygen impermeable seal, the freezing step and sublimation step may be conveniently be conducted in lyophilisation apparatus. In such embodiments, the process of the invention includes the step of loading the sealed receptacle into the lyophilisation apparatus. In certain embodiments, the process of the invention includes the step of opening the sealed receptacle prior to or during the sublimation step.

In alternative embodiments, the step of freezing the lyophilisation medium under anaerobic conditions may be carried out in a first apparatus (e.g. a freezing apparatus) while the sublimation step may be carried out in a second apparatus (e.g. a lyophilisation apparatus) in which supplementary freezing steps may be carried out as part of the freeze-drying process. In such embodiments, the process of the invention comprises the step of transferring the frozen lyophilisation medium from the first apparatus to the second apparatus. In embodiments in which the frozen lyophilisation medium is provided in a receptacle, this step could be performed by loading the receptacle into the lyophilisation apparatus.

The frozen lyophilisation medium may be provided in any form, for example the frozen lyophilisation medium may be a powder, pellet or block form.

Advantageously, the inventors have unexpectedly found that the lyophilisation medium comprising anaerobic bacteria frozen under anaerobic conditions has low susceptibility to inactivation by oxygen. This means that it is not essential to maintain the frozen lyophilisation medium under anaerobic conditions prior to and/or during the sublimation step.

Thus, in embodiments in which the sublimation step is carried out in lyophilisation apparatus, the frozen lyophilisation medium can be exposed an atmosphere comprising oxygen at a level of about 100 ppm or higher, about 200 ppm or higher, about 500 ppm or higher, about 1000 ppm or higher, about 2000 ppm or higher, about 5000 ppm or higher, about 10000 ppm or higher, about 20000 ppm or higher or about 50000 ppm or higher, or ambient air. Such exposure of the frozen lyophilisation medium could arise through an oxygen permeable receptacle containing the frozen lyophilisation medium being transferred from anaerobic conditions to such an atmosphere. Alternatively, such exposure could arise through a sealed oxygen impermeable receptacle being opened (e.g. by removal of a closure of an opening (e.g. a filling port) in the receptacle and/or removal of a portion of the wall of the receptacle) in such an atmosphere.

Accordingly, in embodiments of the invention, the oxygen level of the environment in which the sublimation step is carried out may be about 100 ppm or higher, about 200 ppm or higher, about 500 ppm or higher, about 1000 ppm or higher, about 2000 ppm or higher, about 5000 ppm or higher, about 10000 ppm or higher, about 20000 ppm or higher or about 50000 ppm or higher. In certain embodiments, the sublimation step may be carried out in ambient air.

The inventors have also found that the operating conditions during the sublimation step do not significantly impact the viability of bacterial cells comprised within the frozen lyophilisation medium, provided that the frozen lyophilisation medium was frozen under anaerobic conditions. Accordingly, in certain embodiments, the temperature to which the lyophilisation medium is exposed during the sublimation step may be about 50° C. or lower, about 30° C. or lower, or about 10° C. or lower. In certain embodiments, the temperature to which the lyophilisation medium is exposed during the sublimation step may be about −30° C. or higher, about −50° C. or higher, about −70° C. or higher, about −100° C. or about −150° C. or higher. In certain embodiments, the temperature to which the lyophilisation medium is exposed during the sublimation step may be between about −150° C. to about 50° C. In certain embodiments, the temperature to which the lyophilisation medium is exposed during the sublimation step may be between about −100° C. to about 30° C. In certain embodiments, the temperature to which the lyophilisation medium is exposed during the sublimation step may be between about −70° C. to about 10° C. In certain embodiments, the temperature to which the lyophilisation medium is exposed during the sublimation step may be between about −50° C. to about 10° C. In certain embodiments, the temperature to which the lyophilisation medium is exposed during the sublimation step may be between about −30° C. to about 10° C.

Additionally or alternatively, in certain embodiments, the pressure at which the sublimation step is carried out may be about 5000 pbar or lower, about 2000 pbar or lower, about 1000 pbar or lower or about 500 pbar or lower. In certain embodiments, the pressure at which the sublimation step is carried out may be about 50 pbar or higher, about 25 pbar or higher, about 10 pbar or higher, or about 0 pbar or higher. In certain embodiments, the pressure at which the sublimation step is carried out may be between about 0 pbar to about 5000 pbar. In certain embodiments, the pressure at which the sublimation step is carried out may be between about 10 pbar to about 2000 pbar. In certain embodiments, the pressure at which the sublimation step is carried out may be between about 25 pbar to about 1000 pbar. In certain embodiments, the pressure at which the sublimation step is carried out may be between about 50 pbar to about 500 pbar.

In embodiments in which the frozen lyophilisation medium is provided in a receptacle, the process of the invention may comprise the step of exposing the frozen lyophilisation medium within the receptacle. This may be done to facilitate the sublimation step, desorption step (if performed) or other steps of the lyophilisation process. For example, a portion of the wall of the receptacle could be removed and/or an opening of a closure (e.g. a filling port) in the receptacle. This step of exposing the frozen lyophilisation medium may be performed prior to loading the receptacle into the lyophilisation apparatus, or following loading of the receptacle into the lyophilisation apparatus and prior to or during the sublimation step.

The sublimation step can be conducted using any technique or apparatus known to those skilled in the art of lyophilisation. For example, the sublimation step may be conducted in lyophilisation apparatus. In embodiments of the invention, the lyophilisation apparatus may be of any size without this impacting on the viability of the cells present in the lyophilisation medium. Thus, in certain embodiments of the invention, the sublimation step is carried out in pilot scale lyophilisation apparatus (e.g. freeze drying apparatus having an operating shelf area of about 0.1 m² or higher, about 0.2 m² or higher, about 0.5 m² or about 2 m² or lower, about 3 m² or lower or about 4 m² or lower). In certain embodiments of the invention, the sublimation step is carried out in commercial scale lyophilisation apparatus (e.g. freeze drying apparatus having an operating shelf area of about 5 m² or higher, about 10 m² or higher, or about 20 m² or higher, or about 50 m² or lower, about 100 m² or lower, about 150 m² or lower, or about 200 m² or lower).

### Desorption Step

As those skilled in the art will recognise, conventional lyophilisation processes typically include a plurality of sublimation steps, for example, a sublimation step and a desorption step. Thus, in certain embodiments of the invention, the process comprises one or more desorption steps.

As with the sublimation step, the oxygen level of the environment in which the desorption step is carried out may be about 100 ppm or higher, about 200 ppm or higher, about 500 ppm or higher, about 1000 ppm or higher, about 2000 ppm or higher, about 5000 ppm or higher, about 10000 ppm or higher, about 20000 ppm or higher or about 50000 ppm or higher. In certain embodiments, the desorption step may be carried out in ambient air.

In certain embodiments, during the desorption step, if carried out, the lyophilisation medium may be exposed to a temperature of about 70° C. or lower, about 50° C. or lower, or about 40° C. or lower. In certain embodiments, during the desorption step, the lyophilisation medium may be exposed to a temperature of about 10° C. or higher, about 0° C. or higher, about −10° C. or higher, or about −20° C. or higher. In certain embodiments, during the desorption step, the lyophilisation medium may be exposed to a temperature of between about −20° C. to about 70° C. In certain embodiments, during the desorption step, the lyophilisation medium may be exposed to a temperature of between about −10° C. to about 50° C. In certain embodiments, during the desorption step, the lyophilisation medium may be exposed to a temperature of between about 0° C. to about 40° C. In certain embodiments, during the desorption step, the lyophilisation medium may be exposed to a temperature of between about 10° C. to about 40° C.

Additionally or alternatively, in certain embodiments, during the desorption step, if carried out, the lyophilisation medium may be exposed to a pressure of about 2000 μbar or lower, about 1000 μbar or lower, about 500 μbar or lower, or about 300 μbar or lower. In certain embodiments, during the desorption step, the lyophilisation medium may be exposed to a pressure of about 50 μbar or higher, about 25 μbar or higher, about 10 μbar or higher, or about 0 μbar or higher. In certain embodiments, during the desorption step, the lyophilisation medium may be exposed to a pressure of between about 0 μbar to about 2000 μbar. In certain embodiments, during the desorption step, the lyophilisation medium may be exposed to a pressure of between about 10 μbar to about 1000 μbar. In certain embodiments, during the desorption step, the lyophilisation medium may be exposed to a pressure of between about 25 μbar to about 500 μbar. In certain embodiments, during the desorption step, the lyophilisation medium may be exposed to a pressure of between about 50 μbar to about 300 μbar.

Preferably, the desorption step is carried out in the same apparatus as the sublimation step.

### Additional Processing Steps

As those skilled in the art of lyophilisation will be aware, there are additional processing steps which are conventionally practiced in freeze drying processes, for example a repressurisation step in which the pressure within the lyophilisation apparatus is reverted to atmospheric pressure (preferably by feeding in an inert gas such as nitrogen). In embodiments of the invention, the process additionally comprises this step.

### Lyophilised Product

Following completion of the process of the invention, a lyophilised product is provided. As demonstrated by the examples which follow, if the viable cell count of the bacteria present in that lyophilised product is compared to the viable cell count of the lyophilisation medium prior to freezing, the loss of viable cells observed is minimal. Thus, in embodiments of the invention, the viable cell count (in CFU/g) in the lyophilised product is no more than 10³ CFU/g, 10² CFU/g or 10¹ CFU/g lower than the viable cell count (in CFU/g) of the lyophilisation medium prior to freezing (excluding moisture).

In some embodiments, the viable cell count (in CFU/g as dry weight) of the lyophilised product is equal to or less than 10³ CFU/g, equal to or less than 10² CFU/g, or equal to or less than 10¹ CFU/g lower than the viability of the lyophilisation medium prior to lyophilisation (CFU/ml).

In some embodiments of the invention, the viable cell count (in CFU/g) in the lyophilised product is no more than a factor of 5 CFU/g, no more than a factor of 4 CFU/g, no more than a factor of 3 CFU/g or no more than a factor of 2 CFU/g lower than the viable cell count (in CFU/g) of the lyophilisation medium prior to freezing (excluding moisture).

In some embodiments, the viable cell count (in CFU/g as dry weight) of the lyophilised product is equal to or less than a factor of 5 CFU/g, equal to or less than a factor of 4 CFU/g, equal to or less than a factor of 3 CFU/g, or equal to or less than a factor of 2 CFU/g lower than the viability of the lyophilisation medium prior to lyophilisation (CFU/ml).

In some embodiments, the reduction in viable cell count in the lyophilised product compared to the viable cell count in the lyophilisation medium prior to freezing (excluding moisture) is 1 log or less, 0.5 log or less, 0.4 log or less, 0.3 log or less, 0.2 log or less, or 0.1 log or less. In a particular embodiment, the reduction in viable cell count in the lyophilised product compared to the viable cell count in the lyophilisation medium prior to freezing is 0.3 log or less (excluding moisture). As shown in the examples, the process of the invention is particularly suitable for maintaining viable cell count in the lyophilised product compared to the viable cell count in the lyophilisation medium prior to freezing.

In order to accurately assess the reduction (e.g. log loss) in viable cell count in the lyophilised product (i.e. in CFU/g) compared to the viable cell count in the lyophilisation medium (i.e. in CFU/ml), the skilled person would appreciate that it is necessary to account for the increased concentration of potentially viable cells in the/g of lyophilised product compared to in the/ml of lyophilisation medium (i.e. caused by the removal of moisture during the process of lyophilisation). The increased concentration of cells in the lyophilised product can be accounted for by determining a so-called theoretical viable cell count (in CFU/g) for the lyophilised product which is calculated as a function of the moisture content and the viable cell count of the lyophilisation medium. The theoretical viable cell count assumes that no loss of viability occurs upon the removal of moisture and therefore corresponds to the maximum possible viable cell count in the lyophilised product after lyophilisation (in CFU/g). By comparing the theoretical viable cell count (in CFU/g) to the real viable cell count (in CFU/g) measured after lyophilisation, it is possible to accurately determine the reduction (e.g. log loss) in viable cell count in the lyophilised product, accounting for the increase in concentration of bacterial cells occurring upon removal of moisture during lyophilisation.

This process of accounting for the loss of moisture between the lyophilisation medium and lyophilised product (i.e. before and after lyophilisation) can be explained with the following illustration. Three lyophilisation media have the same viable cell count (in CFU/ml) prior to lyophilisation, but have different % water contents. In a lyophilisation process in which, for the purposes of this illustration, half of the bacteria perish, the overall reduction (e.g. log loss) in viability should be identical for each lyophilisation media irrespective of their original water content. This is possible, as demonstrated in Table 1, below by calculating the theoretical viable cell count (on the basis of moisture content and viable cell count of the lyophilisation medium) for each of the respective lyophilised products (in CFU/g), and comparing this theoretical viable cell count with the ‘real’ viable cell count of the corresponding lyophilised product (in CFU/g) measured after lyophilisation. Accordingly, the reduction (e.g. log loss) in viability for each of the lyophilisation products is identical irrespective of the original water content. Therefore, the inherent increase in concentration of bacterial cells upon lyophilisation and/or any differences in the moisture content of different lyophilisation media prior to lyophilisation are accounted for in the calculation. This factoring ensures at least that (i) the reduction (e.g. log loss) in viable cell count determined when comparing the viable cell count before lyophilisation (in CFU/ml) and after lyophilisation (in CFU/g) accurately represents the loss of bacterial cell viability during the lyophilisation process and (ii) the reduction (e.g. log loss) in viable cell count determined for lyophilised products from lyophilisation media having different moisture contents can be directly compared.

TABLE 1
Determination of reduction (e.g. log loss) in viable cell count

| | | | | Post- | | | | |
|---|---|---|---|---|---|---|---|---|
| | Pre | | | lyophilisation | | Post- | | |
| | lyophilisation | | | theoretical | | lyophilisation | | |
| | viable cell | Water | | viable cell | log | ‘real’ viable | log | |
| Lyophilisation | count | content | Concentration | count | theoretical | cell count | ‘real’ | log |
| medium | (CFU/ml) | (%) | factor | (CFU/g) | value | (CFU/g)* | value | loss |
| A | 1.00 × 10¹⁰ | 20 | 1.25 | 1.25 × 10¹⁰ | 10.10 | 6.25 × 10⁹ | 9.80 | 0.30 |
| B | 1.00 × 10¹⁰ | 50 | 2 | 2.00 × 10¹⁰ | 10.30 | 1.00 × 10¹⁰ | 10.00 | 0.30 |
| C | 1.00 × 10¹⁰ | 80 | 5 | 5.00 × 10¹⁰ | 10.70 | 2.50 × 10¹⁰ | 10.40 | 0.30 |

*As outlined above, the ‘real’ viable cell count for the purposes of this example assumes that half of the bacteria perish during the lyophilisation process.

In line with the calculations outlined above, the viability before lyophilisation (in CFU/ml) and after lyophilisation (in CFU/g) can be directly compared.

Those skilled in the art will be familiar with methods for conducting viable cell counts, for example plate counts using a spiral plater, e.g. that commercialised under the trade mark easySpiral® Pro.

The lyophilised product can be blended with one or more excipients before being provided in dosage forms. Such excipients can comprise diluents, stabilisers, growth stimulators, fillers, lubricants, glidants and the like. Examples of such suitable excipients can be found in the Handbook of Pharmaceutical Excipients. Acceptable excipients for therapeutic use are well known in the pharmaceutical art.

Exemplary pharmaceutically acceptable excipients which may be blended with the lyophilised product include, but are not limited to, binders, disintegrants, superdisintegrants, lubricants, diluents, fillers, flavours, glidants, sorbents, solubilizers, chelating agents, emulsifiers, thickening agents, dispersants, stabilizers, suspending agents, adsorbents, granulating agents, preservatives, buffers, colouring agents and sweeteners or combinations thereof. Examples of binders include microcrystalline cellulose, hydroxypropyl methylcellulose, carboxyvinyl polymer, polyvinylpyrrolidone, polyvinylpolypyrrolidone, carboxymethylcellulose calcium, carboxymethylcellulose sodium, ceratonia, chitosan, cottonseed oil, dextrates, dextrin, ethylcellulose, gelatin, glucose, glyceryl behenate, galactomannan polysaccharide, hydroxyethyl cellulose, hydroxyethylmethyl cellulose, hydroxypropyl cellulose, hypromellose, inulin, lactose, magnesium aluminium silicate, maltodextrin, methylcellulose, poloxamer, polycarbophil, polydextrose, polyethylene glycol, polyethylene oxide, polymethacrylates, sodium alginate, sorbitol, starch, sucrose, sunflower oil, vegetable oil, tocofersolan, zein, or combinations thereof. Examples of disintegrants include hydroxypropyl methylcellulose (HPMC), low substituted hydroxypropyl cellulose (L-HPC), croscarmellose sodium, sodium starch glycolate, lactose, magnesium aluminum silicate, methylcellulose, polacrilin potassium, sodium alginate, starch, or combinations thereof. Examples of a lubricant include stearic acid, sodium stearyl fumarate, glyceryl behenate, calcium stearate, glycerin monostearate, glyceryl palmitostearate, magnesium lauryl sulphate, mineral oil, palmitic acid, myristic acid, poloxamer, polyethylene glycol, sodium benzoate, sodium chloride, sodium lauryl sulphate, talc, zinc stearate, potassium benzoate, magnesium stearate or combinations thereof. Examples of diluents include talc, ammonium alginate, calcium carbonate, calcium lactate, calcium phosphate, calcium silicate, calcium sulphate, cellulose, cellulose acetate, corn starch, dextrates, dextrin, dextrose, erythritol, ethylcellulose, fructose, fumaric acid, glyceryl palmitostearate, isomalt, kaolin, lactitol, lactose, magnesium carbonate, magnesium oxide, maltodextrin, maltose, mannitol, microcrystalline cellulose, polydextrose, polymethacrylates, simethicone, sodium alginate, sodium chloride, sorbitol, starch, sucrose, sulfobutylether β-cyclodextrin, tragacanth, trehalose, xylitol, or combinations thereof.

Various useful fillers or diluents include, but are not limited to calcium phosphate, dibasic anhydrous, calcium phosphate, dibasic dihydrate, calcium phosphate tribasic, calcium sulphate, cellulose powdered, silicified microcrystalline cellulose, cellulose acetate, compressible sugar, confectioners sugar, dextrates, dextrin, dextrose, fructose, kaolin, lactitol, lactose, lactose monohydrate, magnesium carbonate, magnesium oxide, maltodextrin, maltose, mannitol, microcrystalline cellulose, polydextrose, simethicone, sodium alginate, sodium chloride, sorbitol, starch, pregelatinized starch, sucrose, trehalose and xylitol, or mixtures thereof.

Examples of suitable lubricants include, but are not limited to, magnesium stearate, calcium stearate, zinc stearate, stearic acid, talc, glyceryl behenate, polyethylene glycol, polyethylene oxide polymers, sodium lauryl sulphate, magnesium lauryl sulphate, sodium oleate, sodium stearyl fumarate, DL-leucine, colloidal silica, and others as known in the art.

Various useful glidants include, but are not limited to, tribasic calcium phosphate, calcium silicate, cellulose, powdered, colloidal silicon dioxide, magnesium silicate, magnesium trisilicate, starch and talc, or mixtures thereof.

Pharmaceutically acceptable surfactants include, but are limited to both non-ionic and ionic surfactants suitable for use in pharmaceutical dosage forms. Ionic surfactants can include one or more of anionic, cationic or zwitterionic surfactants. Various useful surfactants include, but are not limited to, sodium lauryl sulphate, monooleate, monolaurate, monopalmitate, monostearate or another ester of olyoxyethylene sorbitan, sodium dioctylsulfosuccinate (DOSS), lecithin, stearic alcohol, cetostearylic alcohol, cholesterol, polyoxyethylene ricin oil, polyoxyethylene fatty acid glycerides, and poloxamer.

Excipients which may be blended with the lyophilised product can comprise a prebiotic. The term “prebiotic” means a non-digestible ingredient that beneficially affects the live biotherapeutic bacteria (LBP) by selectively stimulating the growth and/or activity of one or a limited number of bacteria. Examples of prebiotics include oligosaccharides, fructooligosaccharides and galactooligosaccharides.

In embodiments of the invention, the process comprises the step of preparing a dosage form comprising the lyophilised product. Dosage forms comprising the lyophilised product can be prepared by punching or pressing tablet cores comprising the lyophilised product. In certain embodiments, tablet cores are coated (e.g. enteric coating) to provide tablets. In certain embodiments, the lyophilised product is encapsulated into capsule shells to provide capsules. In certain embodiments, the lyophilised product is provided in sachets and sealing the sachets.

In a particular embodiment of the invention, the lyobuffer does not comprise inulin, cysteine and riboflavin, and the reduction in viable cell count in the lyophilised product compared to the viable cell count in the lyophilisation medium prior to freezing is 0.3 log or less (e.g. 0.1 log or less).

### EXAMPLES

### Example 1—Lyophilisation of an Obligate Anaerobic Bacterium with a Freezing Step Performed Under Anaerobic Conditions

A first concentrated biomass of bacteria from the Bacteroides thetaiotamicron strain deposited under accession number NCIMB 42408 with the international depositary authority NCIMB, Ltd. (Ferguson Building, Aberdeen, AB21 9YA, Scotland) on 3 Dec. 2014 (described more fully in International Patent Publication No. WO2016/203217) was prepared. The concentrated biomass had a viable cell count of around 1×10¹¹ CFU/ml. The concentrated biomass was mixed under an anaerobic atmosphere with a lyobuffer.

The resulting mixture was then filled into an oxygen impermeable receptacle of the type disclosed in International Patent Publication No. WO2019/012512, under anaerobic conditions in an isolator provided with a disposable liner. Prior to filling, the receptacle had been purged of oxygen. Once filling was complete, the filling port was closed to provide an oxygen impermeable seal. The filled receptacle was then removed from the isolator and stored under refrigerated conditions for several hours.

Subsequently, the receptacle (and its contents) were snap frozen by cooling in a liquid nitrogen-cooled freezer to −110° C. for two hours.

A portion of the wall of the receptacle was then removed exposing the frozen material to ambient air. The opened receptacle was then loaded into a conventional lyophiliser and freeze-dried.

A second concentrated biomass also having a viable cell count of around 1×10¹¹ CFU/ml was prepared and mixed with a lyobuffer. The resulting mixture was filled into a commercially available, oxygen permeable, Lyogard® tray under ambient atmosphere, loaded into a conventional lyophiliser and freeze-dried under aerobic conditions.

An assessment of the viable cell count of the obtained lyophilisates was made. The lyophilizate which was obtained via the process in which the biomass/lyobuffer was frozen under anaerobic conditions had a viable cell count of 2×10¹⁰ CFU/g, whereas the lyophilizate obtained via the process in which freezing of the biomass/lyobuffer was not conducted under anaerobic conditions had a viable cell count of 1.3×10⁹ CFU/g.

### Example 2—Lyophilisation of Multiple Obligate Anaerobic Bacteria with a Freezing Step Performed Under Anaerobic Conditions

A concentrated biomass of bacterial strains from a number of obligate anaerobic species (Akkermansia muciniphila, Roseburia hominis, Bacteroides sp, Parabacteroides distasonis, Blautia stercoris, Megasphaera massiliensis and Blautia hydrogenotrophicus) was prepared for lyophilisation in line with the process set out in Example 1.

An assessment of the viable cell count (colony-forming units (CFU) or most probable number (MPN)) of the lyophilised products (CFU/g or MPN/g) was made and compared to the viability of the biomass prior to lyophilisation (CFU/ml or MPN/ml), in particular prior to the step of filling of the oxygen impermeable receptacle with the concentrated biomass. As outlined in the detailed description, the actual reduction (i.e. log loss) in viability is calculated with respect to a theoretical viable cell count, which factors in the loss of moisture content and thus the increase in concentration of bacterial cells that inherently occurs during the process of lyophilisation. The results for each of the bacterial strains is provided in Table 2.

TABLE 2
Viability of anaerobic bacteria before and after lyophilisation

| | | Viability | Viability | |
|---|---|---|---|---|
| Strain | | before | after | Log |
| (MRx and | | filling | lyophilisation | loss in |
| NCIMB) | Species | step | process | viability |
| N/A | Akkermansia | 1.4 × 10¹⁰ | 1.4 × 10¹⁰ | 0 |
| | muciniphila | CFU/ml | CFU/g | |
| MRx0001 | Roseburia | 1.3 × 10¹⁰ | 1.3 × 10¹⁰ | 0 |
| (NCIMB | hominis | CFU/ml | CFU/g | |
| 42383) | | | | |
| MRx0002 | Bacteroides sp | 1.3 × 10¹⁰ | 4.3 × 10⁹ | 0.7 |
| (NCIMB | | CFU/ml | CFU/g | |
| 42408) | | | | |
| MRx0005 | Parabacteroides | 7 × 10¹⁰ | 6.4 × 10¹⁰ | 0 |
| (NCIMB | distasonis | CFU/ml | CFU/g | |
| 42382) | | | | |
| MRx0006 | Blautia | 6 × 10⁹ | 1.0 × 10⁹ | 0.5 |
| (NCIMB | stercoris | CFU/ml | CFU/g | |
| 42381) | | | | |
| MRx0029 | Megasphaera | 2 × 10¹⁰ | 1.2 × 10¹⁰ | 0.1 |
| (NCIMB | massiliensis | MPN/ml | MPN/g | |
| 42787) | | | | |
| MRx1234 | Blautia | 2.7 × 10¹¹ | 2.7 × 10¹¹ | 0 |
| (DSM | hydrogenotrophica | MPN/ml | MPN/g | |
| 14294) | | | | |

The lyophilisation process of the present invention ensured no loss in viability of the majority of the anaerobic bacterial strains tested and only a small loss in viability of the remainder of the anaerobic bacterial strains tested, with the resulting viability of the lyophilizate still within permissible regulatory limits. Accordingly, the process of the present invention can be used to successfully lyophilise bacterial strains from a variety of different obligate anaerobic species while maintaining viability of the bacterial strains post-lyophilisation, and is expected to be suitable for the lyophilisation of any anaerobic bacterial strain.

### Numbered Embodiments

1. A process for preparing a lyophilised product comprising the steps of:

providing a lyophilisation medium comprising anaerobic bacteria

freezing the lyophilisation medium under anaerobic conditions to obtain a frozen lyophilisation medium,

conducting a sublimation step on the frozen lyophilisation medium

collecting a lyophilised product.

2. The process of Embodiment 1, further comprising the step of maintaining the lyophilisation medium under anaerobic conditions until the step of freezing the lyophilisation medium is completed.
3. The process of Embodiment 1 or 2, further comprising the step of filling the lyophilisation medium into a receptacle under anaerobic conditions.
4. The process of Embodiment 3, wherein the step of filling the lyophilisation medium into a receptacle is conducted in an isolator lined with a disposable liner.
5. The process of Embodiment 3 or 4, wherein the receptacle is oxygen impermeable.
6. The process of Embodiment 5, wherein, following the step of filling the lyophilisation medium into the receptacle, the receptacle is closed to provide a sealed, oxygen impermeable receptacle.
7. The process of Embodiment 6, wherein the sealed, oxygen impermeable receptacle is loaded into lyophilisation apparatus, optionally wherein the sublimation step is conducted in the lyophilisation apparatus.
8. The process of Embodiment 7, wherein the process comprises the step of exposing the frozen lyophilisation medium within the receptacle.
9. The process of Embodiment 8, wherein the step of exposing the frozen lyophilisation medium within the receptacle is carried out prior to or during the sublimation step.
10. The process of Embodiment 6, wherein the process comprises the step of exposing the frozen lyophilisation medium within the receptacle, prior to loading the receptacle into lyophilisation apparatus.
11. The process of any one of Embodiments 1 to 10, wherein the step of freezing the lyophilisation medium under anaerobic conditions and the sublimation step are carried out in the same apparatus.
12. The process of any one of Embodiments 1 to 10, wherein the step of freezing the lyophilisation medium under anaerobic conditions and the sublimation step are carried out in separate apparatus.
13. The process of any one of Embodiments 1 to 12, wherein the anaerobic bacteria are obligate anaerobic bacteria.
14. The process of Embodiment 13, wherein the obligate anaerobic bacteria belong to a genera selected from the group consisting of Akkermansia, Roseburia, Bacteroides, Parabacteroides, Blautia, Megasphaera and/or Blautia.
15. The process of Embodiment 13 or 14, wherein the obligate anaerobic bacteria belong to a species selected from the group consisting of Akkermansia muciniphila, Roseburia hominis, Bacteroides sp, Bacteroides thetaiotaomicron, Parabacteroides distasonis, Blautia stercoris, Megasphaera massiliensis and/or Blautia hydrogenotrophica.
16. The process of any one of Embodiments 1 to 15, wherein the lyophilisation medium comprises a lyoprotectant, buffer and/or filler.
17. The process of any one of Embodiments 1 to 16, wherein the lyophilisation medium comprises a lyobuffer, optionally wherein the lyobuffer does not comprise:
(a) inulin;
(b) cysteine;
(c) inulin and cysteine;
(d) inulin and riboflavin; or
(e) inulin, cysteine and riboflavin.
18. The process of any one of Embodiments 1 to 17, wherein the frozen lyophilisation medium is a powder, is a block, or is in pellet form.
19. The process of any one of Embodiments 1 to 18, further comprising the step of blending the lyophilised product with one or more excipients.
20. The process of any one of Embodiments 1 to 19, further comprising the step of preparing a dosage form comprising the lyophilised product.
21. A lyophilised product obtainable from the process of any one of Embodiments 1 to 19 or a dosage form obtainable from the process of Embodiment 20

1.-17. (canceled)

18. A method for lyophilizing a product comprising:
(a) providing a lyophilization medium comprising anaerobic bacteria and a lyobuffer;
(b) freezing the lyophilization medium under anaerobic conditions to obtain a frozen lyophilization medium comprising the anaerobic bacteria and the lyobuffer;
(c) conducting a sublimation step on the frozen lyophilization medium; and
(d) collecting a lyophilized product comprising the anaerobic bacteria and the lyobuffer;
wherein the lyobuffer does not comprise inulin, cysteine, and riboflavin.

19. The method of claim 18, further comprising blending the lyophilized product with one or more excipients.

20. The method of claim 18, further comprising counting a viable cell count in the lyophilized product comprising anaerobic bacteria.

21. The method of claim 20, wherein the viable cell count of the anaerobic bacteria in the lyophilized product is no more than 10³ colony forming units per gram (CFU/g) lower than the viable cell count of the anaerobic bacteria in the lyophilization medium prior to step (b).

22. The method of claim 18, wherein the lyophilization medium is kept under anaerobic conditions until the frozen lyophilization medium comprising anaerobic bacteria is obtained.

23. The method of claim 18, further comprising filing the lyophilization medium into a receptacle under anaerobic conditions prior to step (b).

24. The method of claim 23, wherein the filling the lyophilization medium into a receptacle is conducted in an isolator lined with a disposable liner.

25. The method of claim 23, wherein the receptacle is oxygen impermeable.

26. The method of claim 25, further comprising closing the receptacle to provide a sealed, oxygen impermeable receptable.

27. The method of claim 26, further comprising loading the sealed, oxygen impermeable receptable into a lyophilization apparatus.

28. The method of claim 27, further comprising exposing the frozen lyophilization medium within the receptable to ambient air by removing a portion of the wall of the receptacle prior to or during step (c).

29. The method of claim 26, further comprising exposing the frozen lyophilization medium within the receptacle to ambient air prior to loading the receptacle into a lyophilization apparatus.

30. The method of claim 18, wherein step (b) and step (c) are carried out in the same apparatus or in a separate apparatus.

31. The method of claim 18, further comprising preparing a dosage form comprising the lyophilized product.

32. The method of claim 18, wherein the anaerobic bacteria comprise obligate anaerobic bacteria.

33. The method of claim 32, wherein the obligate anaerobic bacteria comprises bacteria of the genus comprising Akkermansia, Roseburia, Bacteroides, Parabacteroides, Blautia, or Megasphaera.

34. The method of claim 32, wherein the obligate anaerobic bacteria comprises bacteria of the species comprising Akkermansia muciniphila, Roseburia hominis, Bacteroides sp, Bacteroides thetaiotaomicron, Parabacteroides distasonis, Blautia stercoris, Megasphaera massiliensis, or Blautia hydrogenotrophica.

35. The method of claim 18, wherein the one or more excipients comprises mannitol, skim milk, bovine serum albumin (BSA), sucrose, trehalose, glucose, maltose, maltotriose, dextran, maltodextrin, or lactose.

36. The method of claim 18, wherein the lyophilization medium further comprises a lyoprotectant, a buffer, or a filler.

37. The method of claim 18, wherein the frozen lyophilization medium is a powder, a block, or a pellet.
